SEQUENCE LISTING (18 sequences)

SEQ ID NO: 1 - DNA, 50 bp, Human genom
ggtgcgggagcagtaggcgtccgcagagcccgcgggggccggcccagccc

SEQ ID NO: 2 - DNA, 50 bp, Human genom
ggtgcgggagcagtaggcgtccgcagagcccgcgggggccgttccagccc

SEQ ID NO: 3 - DNA, 30 bp, Human genom
gtaggggagcctcgcaggcggcggcggcgg

SEQ ID NO: 4 - DNA, 30 bp, Human genom
gtaagggaacctcgcaggcggcggcggcgg

SEQ ID NO: 5 - DNA, 60 bp, Human genom
cggcggccgggtctggccccgacttcggccgggggcggccgtgactgctgcgcccgcgga

SEQ ID NO: 6 - DNA, 60 bp, Human genom
ctctgccgagggtgcgggagcagtaggcgtccgcagagcccgcgggggccggcccagccc

SEQ ID NO: 7 - DNA, 60 bp, Human genom
gggagccgcgcgggcgaggcgagagccgcactgggaggcgcgccgcggcggccgcgagcc

SEQ ID NO: 8 - DNA, 60 bp, Human genom
caggaaagccttccaggaggggccgggcggcaccgcctcccggctccctcctcccgcgcc

SEQ ID NO: 9 - DNA, 60 bp, Human genom
catcccctccgcctcttcctcctcctcctttcgccgccgcctcctccctcccggatctgt

SEQ ID NO: 10 - DNA, 60 bp, Human genom
gctccagttcagagaaagaagaaaatgtgtgtgtgtggcgagggggagggctcgcactga

SEQ ID NO: 11 - DNA, 60 bp, Human genom
gcagccgctccgcgcctggcaatcggggcagggggtgggcgtctggcgggggcgggggcg

SEQ ID NO: 12 - DNA, 60 bp, Human genom
gaggcggaggcggaggagcgcgcggttgccgggcgggacgggaaccgggtctgcgcgcga

SEQ ID NO: 13 - DNA, 60 bp, Human genom
gccgggcggtgggcgcggacagggccgagggcgcccgggagccgtgtcaggcggcggcga

SEQ ID NO: 14 - DNA, 60 bp, Human genom
ggagcgggcgcgccgggcgcggggagccgtcggcggccgctcgctgcggggacaccccgg

SEQ ID NO: 15 - DNA, 60 bp, Human genom
tgaatgagctctcgccgccaggcgcacggcgtaggggagcctcgcaggcggcggcggcgg

SEQ ID NO: 16 - DNA, 60 bp, Human genom
cggcggctcggggggccaggcgggccgcctgttcccggggaaggaggcgcgggcgtctga

SEQ ID NO: 17 - DNA, 60 bp, Human genom
gcgaggccgggcgcggcggccttctctcacgcgccccccgagccagcgcccgcctcccgc

SEQ ID NO: 18 - DNA, 300 bp, Human genom
gcccgcctctcttctcccgtcggcccagactatccgctcccaccgcgcccccggcccacctggtggccccggccctggccgccgcccccgcggcggttcccggagctcgtcccggacgcgcgcccgggcggcgggggctcggcggccaccgctgcctcgggggagcgagggcgggagggtgtgtgtgcgcgctgtgagcagggggtgccggcggggctgcagcggaggcactttggaagaatgactctggagtccatcatggcgtgctgcctgagcgaggaggccaaggaagcccggcgg

(19) United States
(12) Patent Application Publication
(10) Pub. No.: US 2008/0020385 A1
(43) Pub. Date: Jan. 24, 2008

(54) Use Of A Genetic Modification In The Human Gnaq Gene For Predicting Risk Of Disease, The Course Of Disease, And Reaction To Treatments

(76) Inventors: Ulrich Frey, Essen (DE); Winfried Siffert, Essen (DE)

Correspondence Address: AMSTER, ROTHSTEIN & EBENSTEIN LLP, 90 PARK AVENUE, NEW YORK, NY, 10016, US

(21) Appl. No.: 11/597,233

(22) PCT Filed: May 25, 2005
(86) PCT No.: PCT/EP05/05625

§ 371 Date: Jul. 11, 2007

(30) Foreign Application Priority Data
May 26, 2004 (DE) 10 2004 026 330.2

(51) Int. Cl. C12Q 1/68

(52) U.S. Cl. 435/6

(57) ABSTRACT

In order to determine the risk of disease, the course of a disease, the action of medicaments, the side-effects of medicaments and drug targets, a base substitution is identified in the non-translated region 5′ of the gene for the Gαq sub-unit of human G proteins, preferably the presence of two or three of the polymorphisms GC(−909/−908)TT, G(−382)A or G(−387)A being detected.

### TECHNICAL FIELD OF THE INVENTION

The invention concerns methods to detect the presence of various polymorphisms in the human Gáq gene (GNAQ) for prediction of the risks of disease, the course of diseases and the selection of individually suitable therapy methods.

All cells in the human body possess membrane receptors on their surface, through which all cell functions are controlled. These receptors include the so-called heptahelical receptors for hormones, neurotranmitters and chemokines. In addition, there are many receptors for growth factors and receptors with intrinsic tyrosine kinase activity, for example, receptors for insulin, insulin-like growth factor, epidermal growth factor, platelet-derived growth factor and many more. There are in addition many receptors which are responsible for the regulation of hematopoesis, e.g. the receptor for erythropoietin. For example, cell growth, motility, gene expression, apoptosis and chemotaxis are controlled by receptors of this sort. These receptors transmit their signals into the interior of the cell through the activation of the so-called heterotrimeric G proteins. These G proteins consist of a large family of different α-, β- and γ-subunits. 5 β-Subunits, 13 γ-subunits and more than 20 α-subunits are currently known, which are coded by different genes (Farfel Z et al. 1999). Many different heterotrimeric G proteins are formed by the combination of these different α-, β- and γ-subunits.

The isoform combination then determines which heterotrimer can be activated by a defined receptor. The βγ-subunits should be regarded as a functional monomer. In the resting state, the α-subunit has bound GDP (FIG. 1). After activation of a coupling receptor, the α-subunit releases GDP in exchange for GTP and the βγ-subunits are dissociated from the α-subunits. Both the free α-subunits and the βγ-subunits can direct the activity of a variety of different effectors. These include, for example, ion channels, adenyl cyclase, the PI3-kinase, various MAP-kinases etc. The α-subunits possess intrinsic GTPase activity, which hydrolyses bound GTP to GDP after activation. The βγ-subunits then re-associate with the α-subunit, thus ending the activation cycle. The heterotrimer is then available for a renewed activation cycle (Bourne 1997). FIG. 1 depicts the G protein cycle. The activation of G proteins of this sort is a decisive step in cell activation. Because of the overwhelming importance of G proteins, it is immediately evident that mutations or genetic polymorphisms in genes which code for G proteins must have a sustained effect on the activability of cells, if these mutations influence the function or expression of G protein subunits. This will then have a decisive effect on the risks of disease or on the course of diseases. In addition, the response to the therapy of diseases, either from drugs or from other measures, such as radiation, diets, operations, invasive treatment etc., depends on the activability of G proteins.

Significance of the Gαq-Subunit

The Gαq-subunit is expressed in all cells of the human body. The effects of its stimulation include the activation of phospholipase C, leading to an increase in intracellular Ca²⁺ concentration (FIG. 1). For example, in this way Ca²⁺-dependent processes can be activated. In addition, Gαq can regulate the activity of ion channels, e.g. of potassium or calcium channels. Almost all known receptors couple to G\acute{α}q, e.g. the receptors for acetylcholine, adenosine, adrenaline, angiotensin, bradykinin, endothelin, histamine, noradrenaline, P₂-purinergic receptors, opioids, dopamine, epidermal growth factor, FSH, VIP, thyroliberin, glucagon, vasopressin, histamine and many more. After stimulation of Gαq-coupled receptors, apoptosis is induced in many cell types, so that there is a connection with tumor diseases and their course and response to therapy and also with inflammatory diseases and their course and response to therapy. In addition, a variety of metabolic pathways are regulated by Gαq. In animal experiments or on cellular level, modifications of the expression of Gαq (overexpression or missing expression) lead to a number of disease conditions or phenotypes:
1. Overexpression of Gαq in the heart results in hypertrophy, cardiac insufficiency and apoptosis;
2. Constitutively active Gαq induces apoptosis via the protein kinase C pathway;
3. Knockout of Gαq inhibits thrombocyte aggregation, leads to ataxia and interferes with motoric coordination;
4. Knockout of Gαq results in obesity, and Gαq participates in insulin signal transduction;
5. Constitutively active Gαq subunits are oncogenic (De at al., 1992), and participate in the regulation of glucose metabolism.

These few examples already demonstrate that function-altering Gαq mutations or over- or underexpression of the protein lead to various diseases and/or functional disturbances in humans as well.

### SUMMARY OF THE INVENTION

The invention is based on the object to find polymorphisms and to clarify their physiological or patho-physiological role, and therefore
a. To provide function modifying genomic polymorphisms and haplotypes in the GNAQ gene which either lead to amino acid exchange, or
b. influence the splicing behavior, or
c. which lead to modification in protein expression or to modification of the expression of splice variants, or
d. which are suitable for the identification and/or validation of additional polymorphisms or haplotypes in the GNAQ gene;
e. To provide nucleotide exchanges and haplotypes which are suited in general for the prediction of disease risks and the course of diseases;
f. To provide nucleotide exchanges and haplotypes which are suited in general for the prediction of the response to drugs and of side-effects;
g. To provide nucleotide exchanges and haplotypes which can in general predict the action of other forms of therapy (radiation; warmth, heat, cold, movement) etc.

Because of the fundamental significance of Gαq for signal transduction, polymorphisms or haplotypes of this sort are suited in general for the prediction of the risks of disease or the courses of disease for all diseases and for the prediction of the response to therapy or failure of therapy or undesired side-effects for all pharmacological or non-pharmacological therapies.

This object is solved by a method to identify a risk of disease, a course of disease, of drug effects, drug side-effects and drug targets, associated with a base substitution in the GNAQ gene encoding the Gαq subunit of human G proteins, by identifying a base exchange (polymorphism) in the 5′ non-translated region of the gene for the Gαq subunit of human G proteins.

Another object of the invention is a gene test, comprising a probe for identification of one or more polymorphisms in the 5′ non-translated region of the GNAQ gene.

### DESCRIPTION OF THE FIGURES

FIG. 1—The Gαq signaling pathway. The diagram shows how the Gαq pathway is connected with various signal transduction components after receptor stimulation, including ion channels, transcription factors and synthesis of eicosanoids. PLC, phospholipase C, IP3, inositol triphophate; PKC, protein kinase C; PLA2, phospholipase A2, AA, arachidonic acid; MLCK, myosin light chain kinase; CaM, calmodulin; p42/p44; p42 and p44 MAP kinase.

FIG. 2—Intron/exon structure of human GNAQ and location of the GC(−909/−908)TT polymorphism (not to scale).

FIG. 3—Putative binding sites for transcription factors on the GNAQ gene promoter. The numbers on the right represent the position relative to ATG, the numbers on the left refer to the transcription starting point.

FIG. 4—Results of the electrophoretic mobility shift assays (EMSA) with constructs containing the genotypes GC or TT in the GNAQ promoter. After addition of nuclear extracts, an increased binding of nuclear protein to the GC construct containing another binding site for the transcription factor SP1 may be observed. The binding is specifically inhibited by an anti SP1 antibody or in presence of a displacing SP1 oligonucleotide.

FIG. 5—Constructs for determining the promoter activity through secreted alkaline phosphatase (SEAP). In the left part of the Figure, the constructs used for the reporter assay are described. The right part of the Figure shows the SEAP activity measured 24 h after transfection of HEK cells and smooth muscle cells of rat aorta (A-10)

FIG. 6—Genotype-dependent activity of the GNAQ promoter. The promoter construct −798/+89 with either the GC or the TT genotype was transfected into HEK cells. Secretion of alkaline phosphatase was determined after stimulation with serum or angiotensin. An enhanced activity of the GC genotype promoter can be observed.

FIG. 7—GC(−909/−908)TT polymorphism-dependent tissue expression of GNAQ mRNA. The Gαq/â-actin mRNA ratio is shown.

FIG. 8—Protein/DNA ratio in human heart during atrial fibrillation (AF) and sinus rhythm (SR) and dependence of the protein/DNA ratio on the GC (−909/−908) TT polymorphism.

FIG. 9—GNAQ GC(−909/−908)TT polymorphism and Ca²⁺ increases in skin fibroblasts after stimulation with bradykinin. In cells from subjects with at least one GC allele, stronger increases of the free cytoplasmic Ca²⁺ concentrations may be observed.

FIG. 10—GNAQ GC(−909/−908)TT polymorphism and circulation parameters in healthy individuals. Cardiac stroke volume (left) and total peripheral resistance (right) are shown in relation to the genotype.

FIG. 11—GNAQ GC(−909/−908)TT polymorphism and disease progression in patients with chronic cardiac insufficiency. The time from initial diagnosis to heart transplantation is shown as a measure for disease progression. The disease progress is more favorable in presence of the GC/GC genotype.

FIG. 12—Expression of GNAQ mRNA in adipose tissue relative to the GC (−909/−908) TT polymorphism. The Gαq/â-actin mRNA ratio is shown.

FIG. 13—Inhibiting effect of insulin on isoprenaline-induced lipolysis relative to the GC (−909/−908) TT polymorphism. Glycerol release as a marker for lipolysis is shown.

FIG. 14—BMI, HOMA-IR and insulin concentration relative to the GC (−909/−908) TT polymorphism.

FIG. 15—GNAQ GC(−909/−908)TT polymorphism and serum cholesterol in healthy individuals.

FIG. 16—GNAQ GC(−909/−908)TT polymorphism and disease progression in patients with CLL. The time from initial diagnosis to therapy initiation is shown as a measure for disease progression. The disease progress is more favorable in presence of the TT/TT genotype.

FIG. 17A—GNAQ GC(−909/−908)TT polymorphism and time to metastasis in patients with urinary bladder carcinoma.

FIG. 17B—GNAQ GC(−909/−908)TT polymorphism und time to tumor progression in patients with urinary bladder carcinoma.

FIG. 17C—GNAQ GC(−909/−908)TT polymorphism and survival in patients with urinary bladder carcinoma.

FIG. 18—GNAQ GC(−909/−908)TT polymorphism vasoconstriction after skin injection of noradrenaline, angiotensin, or endothelin. The modification of skin circulation after the injection is shown.

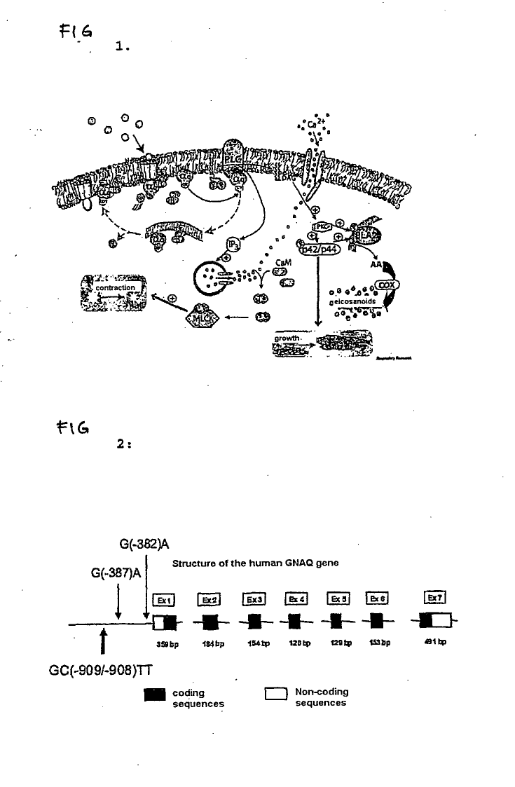

### DETAILED DESCRIPTION OF THE INVENTION

In the method according to the invention, one or more polymorphisms in the 5′ non-translated region of the GNAQ gene are identified. Polymorphisms that are preferably identified according to the method according to the invention, comprise a GC(−909/−908)TT, a G(−382)A, or a G(−387)A polymorphism, or two or three of these polymorphisms.

According to a preferred embodiment the gene test according to the invention contains one or more probes for identification of one or more of the polymorphisms GC(−909/−908)TT, G(−382)A, or G(−387)A in the 5′ non-translated region of the GNAQ gene.

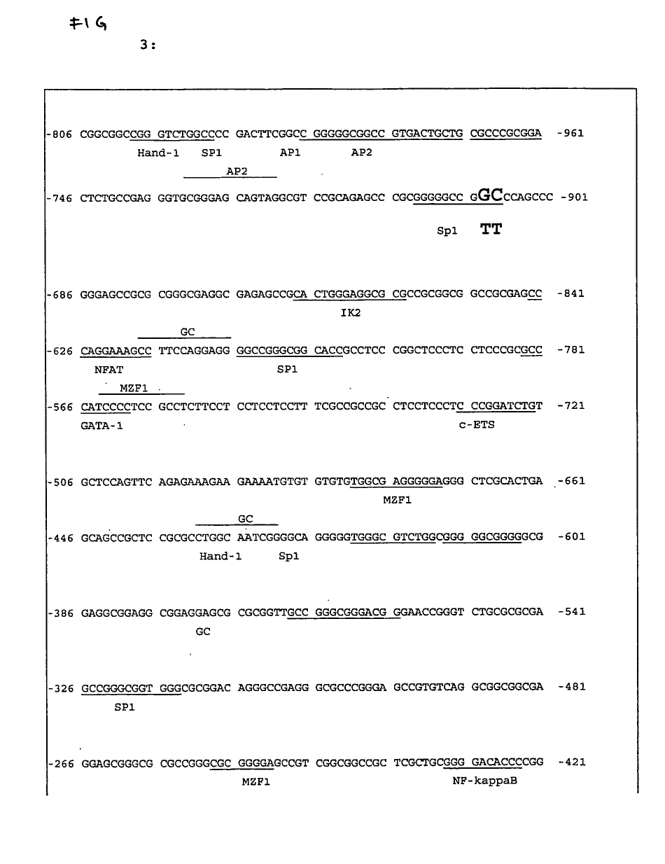

The human Gαq gene (GNAQ) is located on chromosome 9q21 (Gen-Bank Accession Number NM_{—}002072, FIG. 2). The relevant element of the invention is the identification of the gene polymorphisms GC(−909/−908)TT, G(−382)A, or G(−387)A lying before exon 1 in the promoter of the gene, which have been identified by systematic sequencing of human DNA samples. For this, genetic sequences lying before exon 1 in GNAQ were amplified by PCR and sequenced with the method according to Sanger. The PCR reactions may be performed according the so-called slowdown method, and the respective PCR and/or sequencing primers may be selected according to an established algorithm described by Bachmann et al., Pharmacogenetics 13 (12) 759-76, December 2003. The disclosure of this publication is referred to for the purpose of this invention.

The polymorphisms identified according to the invention display a substitution of guanine by thymine G(−909)T in the promoter region on position −909, whereas at the same time cytosine on position −908 is replaced by thymine C(−908)T. The replacements G(−909)T and C(−908)T are always present concomitantly, so that the genotypes TT/TT, GC/GC and TT/GC result. In the G(−382)A polymorphism, guanine on position −382 is replaced by adenine, in the G(−387)A polymorphism, guanine on position −387 is replaced by adenine. The corresponding partial sequences for the GC(−909/−908)TT polymorphism therefore are:

GGTGCGGGAG CAGTAGGCGT CCGCAGAGCC CGCGGGGGCC GGCCCAGCCC - 901

and

GGTGCGGGAG CAGTAGGCGT CCGCAGAGCC CGCGGGGGCC GTTCCAGCCC 901

The partial sequences for the G(−382)A and G(−387)A polymorphisms are:

GTAGGGGAGC CTCGCAGGCG GCGGCGGCGG - 361

and

GTAAGGGAAC CTCGCAGGCG GCGGCGGCGG - 361

The numbering of these SNPs was performed in such a way that the nucleotide A of the start codon ATG was assigned the number +1. As in accordance with the convention there is no number 0, the nucleotide lying before the A of start codon ATG is assigned the number −1.

The detection of these SNPs in the sense of their use in accordance with the invention can be performed with any procedure familiar to the expert, e.g. direct sequencing, PCR followed by restriction analysis, reverse hybridization, dot-blot or slot-blot procedure, mass spectrometry, Taqman® or Light-cycler® technology, Pyrosequencing®, Invader® technology, Luminex procedure etc. In addition, these gene polymorphisms may be simultaneously detected after mulitplex-PCR and hybridization on a DNA-chip.

Distribution of the GC(−909/−908)TT, G(−382)A and G/−387)A Polymorphisms in Different Ethnic Groups, and Use of These Genotypes in the Identification of Additional Relevant Polymorphisms and Haplotypes

For this purpose, different DNA samples from Caucasians, black Africans and Chinese were genotyped. The results are given in the following table.

| Genotype | White Caucasians | Chinese | Black Africans |
|---|---|---|---|
| n | 187 | 109 | 147 |
| TT/TT | 40 | 4 | 2 |
| TT/GC | 97 | 34 | 44 |
| GC/GC | 50 | 71 | 101 |
| % GC | 52.7 | 80.7 | 83.7 |

This genotype distribution is highly significantly different in the chi² test, with a chi 86.1 and p<0.0001. The GC genotype frequency is greatest in black Africans.

It can be deduced from these distributions that, from the evolutionary point of view (relative to Caucasians), the “original state” is GC(−909/−908). Differences of this sort in genotype distribution in different ethnic groups generally indicate that the associated phenotypes were of importance for evolution and that they brought the carriers of these phenotypes certain advantages. The expert is aware that different genotype distribution in different ethnic groups is an indication that even today certain genotypes or haplotypes are associated with certain diseases or with certain physiological or pathophysiological ways of reacting or responding to therapy, e.g. with drugs.

GNAQ G(−382)A

| Genotype | Caucasian | | Black Africans | | Chinese | |
|---|---|---|---|---|---|---|
| GG, (n, %) | 123 | (83.7) | 66 | (91.7) | 109 | (100.0) |
| GA, (n, %) | 22 | (15.0) | 6 | (8.3) | 0 | (0.0) |
| AA (n, %) | 2 | (1.4) | 0 | (0.0) | 0 | (0.0) |
| Total | 147 | | 72 | | 109 | |

| | | | |
|---|---|---|---|
| % G | 91.2% | 95.8% | 100.0% |

This genotype distribution is highly significantly different between Caucasians and Chinese in the chi² test, with chi 18.30 and p<0.0001. The G(−382) allele frequency (% G) is highest in Chinese, followed by Black Africans and Caucasians.

GNAQ G(−387)A

| Genotype | Caucasians | | Black Africans | | Chinese | |
|---|---|---|---|---|---|---|
| GG, (n, %) | 131 | (89.1) | 67 | (93.1) | 107 | (98.2) |
| GA, (n, %) | 16 | (10.9) | 5 | (6.9) | 0 | (1.8) |
| AA (n, %) | 0 | (0.0) | 0 | (0.0) | 0 | (0.0) |
| Total | 147 | | 72 | | 109 | |

| | | | |
|---|---|---|---|
| % G | 94.6% | 96.5% | 99.1% |

This genotype distribution is significantly different between Caucasians and Chinese in the chi² test, with chi 7.7 and p<0.01. The G(−387) allele frequency (% G) is highest in Chinese, followed by Black Africans and Caucasians.

A further analysis finds a coupling disequilibrium between all three polymorphisms in Caucasians. Coupling disequilibrium means that allele combinations (haplotypes) occur which are clearly statistically more frequent or rarer in combination than would be expected on the basis of their frequency.

The following table shows the distribution of G(−387)A genotypes for Caucasians stratified for G(−382)A genotypes.

| | G(−387)A | | | |
|---|---|---|---|---|
| G(−382)A | GG | GA | AA | Total |
| GG | 110 | 13 | 0 | 123 |
| GA | 19 | 3 | 0 | 22 |
| AA | 2 | 0 | 0 | 2 |
| Total | 131 | 26 | 0 | 147 |

There is no statistically significant association between these polymorphisms. There is no coupling disequilibrium between the G(−382)A and the G(−387)A SNP. The following table shows the distribution of G(−382)A genotypes for Caucasians stratified for GC(−909/−908)TT genotypes.

| | GC(−909/−908)TT | | | |
|---|---|---|---|---|
| G(−382)A | GCGC | GCTT | TTTT | Total |
| GG | 30 | 57 | 33 | 120 |
| GA | 10 | 10 | 0 | 20 |
| AA | 2 | 0 | 0 | 2 |
| Total | 42 | 67 | 33 | 142 |

Coupling analyses show a significant coupling disequilibrium between the GC(−909/−908)TT and the G(−382)A SNP (chi²=18.3, p=0.01). The following table shows the distribution of G(−387)A genotypes for Caucasians stratified for GC(−909/−908)TT genotypes.

| | GC(−909/−908)TT | | | |
|---|---|---|---|---|
| G(−387)A | GCGC | GCTT | TTTT | Total |
| GG | 31 | 62 | 33 | 126 |
| GA | 11 | 5 | 0 | 16 |
| AA | 0 | 0 | 0 | 0 |
| Total | 42 | 67 | 33 | 142 |

Coupling analyses show a significant coupling disequilibrium between the GC(−909/−908)TT and the G(−387)A SNP (chi²=14.1, p=0.01). In summary, there is a significant coupling disequilibrium between the GC(−909/−908)TT and both, the G(−382)A and the G(−387)A SNP. On the other hand, the G(−382)A and the G(−387)A SNP are inherited independently from each other.

Another object of this invention is that these new polymorphisms can be used to detect and to validate additional relevant genomic gene modifications in GNAQ or in neighboring genes, which, for example, are in coupling disequilibrium with genotypes in the GNAQ gene. These can also be genes which are also on chromosome 9, but at greater distances from the GNAQ gene. The procedure for this purpose was as follows:
1. For certain phenotypes (cellular properties, disease states, disease courses, response to drugs etc.) an association between the polymorphisms GC(−909/−908)TT or G(−382)A or G(−387)A is first established.
2. For newly detected gene modifications in the GNAQ or neighboring genes, it is investigated whether existing associations were enhanced or weakened by using the genotypes or haplotypes described above.

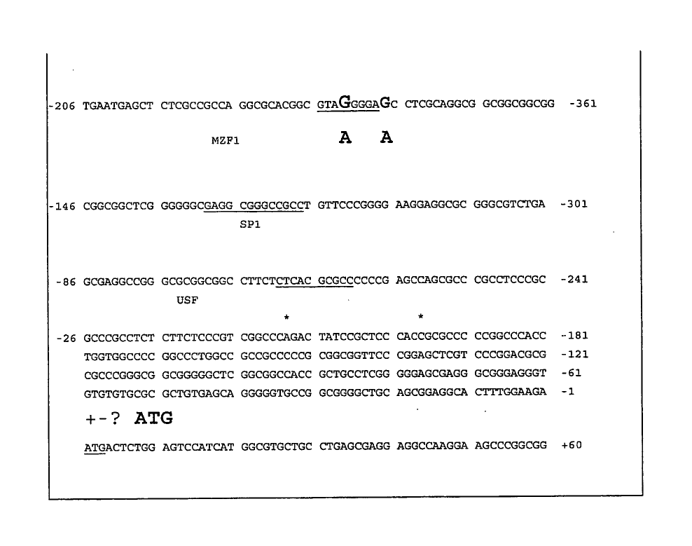

Functional Significance of the GC(−909/−908)TT Polymorphism It was investigated which functional modifications can be assigned to gene modifications in the GNAQ gene. For example, correlation with alternative splicing, tissue-specific expression or overexpression of the Gαq-protein in dependence on the GC(−909/−908)TT polymorphism would be conceivable. For this purpose it was analyzed using a computer program if the identified nucleotide changes are capable of affecting the binding of transcription factors. Transcription factors bind to specific consensus sequences, and can enhance or reduce the promoter activity, resulting in an enhanced or reduced transcription of the gene, and therefore resulting in a higher or lower level of expression of the encoded protein. As shown in FIG. 3, the GC(−909/−908)TT polymorphism is located in a consensus sequence of the binding site for transcription factor SP1 the binding ability of which may be affected by the polymorphism. This effect refers to the (−909/−908)TT genotype. The occurrence of this genotype results in the absence of a SP1 binding site to its consensus sequence: GGGGCGGGGC. In order to study this effect experimentally, a so-called EMSA (electrophoretic mobility shift assay) is conducted. In this study, short nucleic acid fragments containing the polymorphism are incubated with cellular nuclear extracts. Transcription factor proteins present in these extracts now bind to the nucleic acid fragments with varying intensity. Finally, DNA binding is made visible on a X-ray film. A strong binding results in an intensive band. FIG. 4 shows the result of this study with specific constructs containing either the TT or the GC genotype. The stronger intensity of the GC construct band demonstrates a stronger binding of a transcription factor to this region. The disappearance of this band by addition of an SP1 antibody and the displacement of the binding by a commercial SP1 oligonucleotide show that the binding transcription factor is SP1.

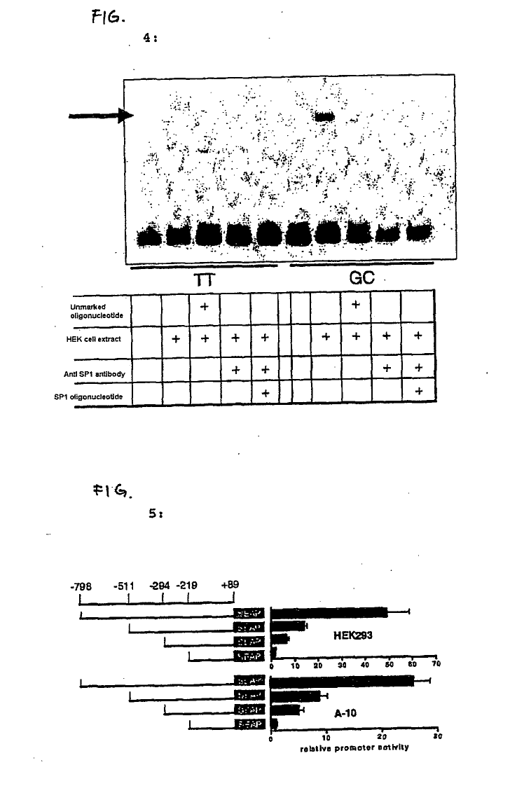

For functional detection of a modified promoter activity in relation to specific genotypes, various fragments of the promoter with the GC or the TT genotype were cloned into the pSEAP vector to quantify the promoter activity after expression of the vector in HEK cells in a so-called reporter assay (FIG. 5). For this assay, constructs are cloned before a gene encoding the secreted alkaline phosphatase (SEAP). If the construct has promoter activity, the SEAP gene will be transcribed more, and the increased secretion of alkaline phosphatase into the cell culture medium can be measured. As shown in FIG. 5, the promoter activity is highest in construct −798/+89 (the numbers refer to the transcription starting point, which is −214 in the GNAQ gene). Since the polymorphism is located in this region (−697/−695 relative from the transcription starting point) showing the highest reporter activity, and furthermore a transcription factor binding site is influenced by the nucleic acid exchange, it is now studied if stimulation of HEK cells has an effect on the reporter activity of these constructs. For this, construct −798/+89 was transfected into HEK cells, together with either the GC(−909/−908) or the TT(−909/−908) polymorphism, and the cells were stimulated with serum or angiotensin II (FIG. 6). In constructs with the GC genotype, the stimulation with serum or 10 nM angiotensin II leads to a significantly (p<0.05) two to four times increased promoter activity compared to the TT genotype. The GC polymorphism in the promoter of the GNAQ gene therefore leads to an increased promoter activity, and thus to an increased expression of the Gαq protein. In order to verify if this regulation also occurs in vivo, Gαq expression was analyzed on mRNA level by real time-PCR.

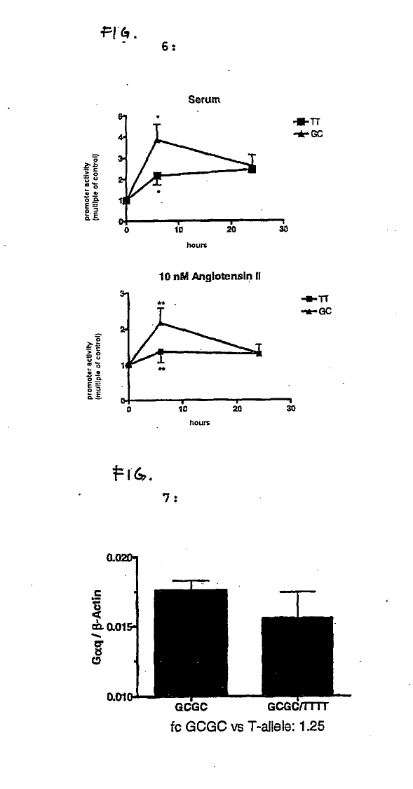

For this, mRNA was isolated from human surgical tissue during heart surgeries and transcribed into cDNA using reverse transcriptase. The procedure is familiar to the expert. The level of expression was then determined with real time-PCR (Taqman procedure) and corrected for the level of expression of the housekeeping gene β-actin. The results are shown in FIG. 7. The GCGC genotyope leads to an increase of G?q transcription by at least 25% compared to the T allele.

In order to further analyze if the GC(−909/−908)TT polymorphism may contribute to the development of cardiac hypertrophy, the protein content was determined compared to the DNA content of heart samples from patients with chronic atrial fibrillation and patients with sinus rhythm. FIG. 8 shows that the relative cellular protein content in samples from patients with atrial fibrillation is increased compared to samples from patients with sinus rhythm. Since the frequency of cardiac hypertrophy in patients with chronic atrial fibrillation is increased compared to patients with sinus rhythm, the protein/DNA index is a marker for cardiac hypertrophy. FIG. 8 shows the protein/DNA index, divided according to the GC(−909/−908)TT polymorphism. This Figure shows that in samples from patients with chronic atrial fibrillation and also in samples from patients with sinus rhythm, the protein/DNA index is highest for GC/GC genotypes.

This proves that there are gene modifications in the GNAQ gene resulting in a modified expression of Gαq in heart tissue, and also possibly resulting in a modification of the protein quantity in the heart. This may be the GC(−909/−908)TT polymorphism or polymorphisms that are in coupling disequilibrium with it (e. g. the G(−382)A or the G(−387)A polymorphism). Thus, it is also a component of the here described invention to quantify Gαq expression on mRNA level or on protein level, to associate it with known GNAQ polymorphisms, and to identify and to validate new, even better suited polymorphisms. The here presented results of a genotype-dependent expression of Gαq and a modified total protein content in the human heart are of considerable significance. In transgenic animals, overexpression of Gαq leads to increased apoptosis of cardiac myocytes as a cause of cardiac insufficiency (Adams and Brown, 2001; Mende et al., 1998). The same effects would therefore be expected in humans overexpressing the Gαs protein because of genetic modification of the GNAQ. This would be expected to lead to increased cardiovascular risk. This includes increased risks of obesity, hypertension, stroke, coronary heart disease, myocardial infarction, pre-eclampsia etc. Moreover, we would expect differences in the response to substances for which Gαq activates the receptors or to substances which indirectly induce agonists of which the activity is mediated through Gαq. The altered tendency to apoptosis can influence the course of a variety of diseases (e.g. tumor and immune diseases) and determine the response to drugs.

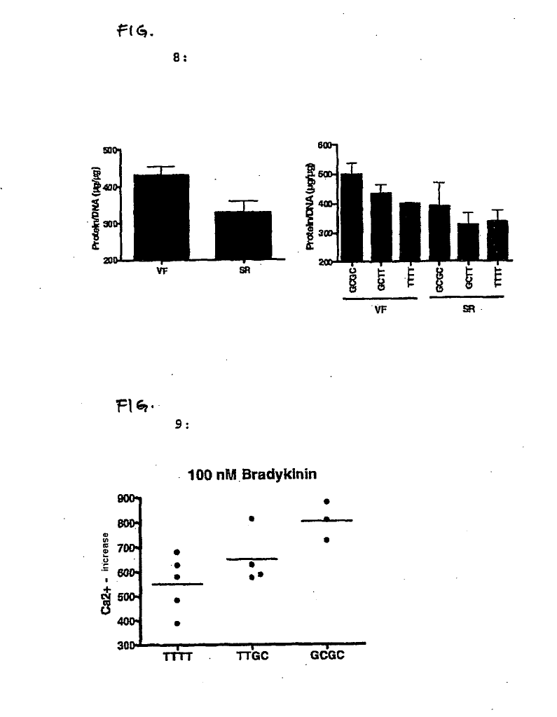

In addition, the overexpression of Gαq in GC allele carriers leads to a consecutively increased signal transduction after stimulation of cells with agonists the signal transduction of which comprises the Gαq protein. The detection is performed in human skin fibroblasts that have been loaded with the Ca²⁺ indicator Fura-2, after stimulation with the hormone bradykinin for which is known that Gαq participates in its effect (FIG. 9). It can be seen clearly that the level of the increase of the intracellular Ca²⁺ concentration increases as the number of GC alleles increases. Thus, the identification of gene modifications in the GNAQ gene is, in principle, suited to predict the level of Gq activation and therefore the efficiency of signal transduction via Gq-coupled receptors.

Use of a Gene Modification in GNAQ to Predict the Risks of Disease and the Course of Diseases

As the Gαq-subunit has a key role in cell activation, it is an essential component of the invention that in general the risks of diseases and the course of diseases can be predicted by using the gene modifications.

Human heterotrimeric G proteins are composed of the subunits α, β and γ. A series of isoforms of these are known, coded by different genes. For example, there are 13 different γ-isoforms (γ₁-γ₁₃), at least 5 different β-isoforms (β₁-β₅) and many different α-isoforms (αₛ (short and long), αₒ, αᵢ₁₋₃, α_{q}, α₁₁₋₁₆, α_{olf} etc.) As G proteins are known to play a central role in controlling the function of all human cells, independently of which cell receptors are activated, it should be expected directly that the course of a variety of totally different diseases would be influenced by a genetically determined, enhanced activability of G proteins. Particularly with the variety of functions of G proteins, mutations modifying function attain extraordinarily great significance and are of great predictive power. This is in contrast to mutations in other genes, e.g. those coding for hormones or hormone receptors.

These means that genetic modifications in proteins which are expressed in all human body cells and which collect all incoming hormonal signals centrally, and which thus regulate cell functions, can decisively influence all physiological and pathophysiological processes or at least modulate them. In addition, responses to drugs will also be decisively influenced. This concerns both desired and undesired drug effects.

It has been repeatedly postulated in the scientific literature that functional modifications in G proteins have a persistent influence on a variety of diseases or on the course of a variety of diseases. These gene modifications may be mutations leading to structural changes in G protein subunits, which may for example modify ability to be activated by a receptor; they may concern the enzymatic GTPase activity or affect the dimerization of the βγ-subunits. In addition, such modifications may alter the composition of heterotrimeric G proteins. Moreover, the level of expression of such G protein subunits may be changed or splice variants with modified function may occur (Farfel et al.; 1999, Iiri and Bourne, 1998; Iiri et al., 1998; Spiegel 1.999; Spiegel, 1997; Spiegel, 1996).

The following can be concluded from the above examples:
1. Gene modifications in genes which code for ubiquitously expressed proteins influence a variety of diseases or cause a variety of risks of disease.
2. G proteins control almost all processes in signal transduction in the human body.
3. On the one hand, it is obvious from the cited literature that it is generally assumed that mutations and polymorphisms in the genes coding for G proteins can evoke such diseases. On the other hand, a connection between genomic mutations in the GNAQ gene for the Gαq-subunit of heterotrimeric G proteins and risks of disease has neither been described nor assumed in the literature.

All diseases which are accompanied by a gene modification in GNAS and which, for example, are determined by a modification in the level of expression of the Gαq protein, can be named as follows:
1. Cardiovascular diseases. These particularly include hypertension, stroke, coronary heart disease and myocardial infarction, heart failure, pre-eclampsia or gestosis.
2. Enocrinological and metabolic diseases. These include obesity, metabolic syndrome, type 2 diabetes mellitus, gout, osteoporosis, thyroid diseases such as hyperthyroidism and hypothyroidism and Basedow disease, hyper- and hypoparathyroidism, Cushing's disease, hyper- and hypoaldosteronism and many more.
3. Psychiatric diseases, such as depression, schizophrenia, alcoholism and anxiety disorder, phobias, neuroses.
4. Neurological diseases such as Parkinson's disease, multiple sclerosis, epilepsy.
5. Dermatological diseases, such as psoriasis, neurodermitis.
6. Tumor diseases.

Use of a Gene Modification in the Human GNAQ-Gene to Predict the Risks of Cardiovascular Disease

In transgenic animals that overexpress Gαq in the heart, an increased tendency for cardiac hypertrophy, heart failure and apoptosis is observed. Therefore, it can be assumed that an increased expression of Gαq in humans, as it is the case with the GC genotype of the GC(−909/−908)TT polymorphism, leads to an increased cardiovascular risk. As shown in FIG. 10, an increased cardiac stroke volume together with a reduced peripheral resistance is already found in young men carrying the GC genotype. As generally known, this is an indication of a hyperdynamic circulatory situation that is associated with an increased risk of hypertension and cardiac hypertrophy. In elderly people we find a significant dependency of the left ventricular mass index from the GNAQ GC(−909/−908)TT polymorphism (p<0.05):

| | Genotype | | |
|---|---|---|---|
| | GC/GC | GC/TT | TT/TT |
| LVMI (g/m²) | 82.4 ± 0.9 | 77.3 ± 0.3 | 73.5 ± 1.2 |

Therefore, there is an increased risk for GC genotypes for left heart hypertrophy with an increased risk for atrial fibrillation, other dysrhythmias and sudden cardiac death.

Use of a Gene Modification in the Human GNAQ-Gene to Predict the Risks of Heart Failure in Case of Chronic Cardiac Insufficiency

The role of Gq in the development of cardiac hypertrophy has been characterized sufficiently well using studies with transgenic mice. However, the course of this disease is influenced by several factors: ejection fraction of the heart, neurohumoral activability of the heart, physiological condition and genetic variability (Molkentin and Dorn II, 2001; Givertz et al., 2004). These and other factors determine the disease course which can lead to death or to heart transplantation due to chronic cardiac insufficiency. Therefore, it can be assumed that a modified activability of the Gq gene through neurohumoral activation, e.g. by angiotensin II, as it is the case with the GC(−909/−908)TT polymorphism, may ultimately influence the disease course in cardiac insufficiency. In patients with chronic cardiac insufficiency due to cardiomyopathy the enhanced neuro-humoral activability represents itself as an improved ejection performance of the heart in homozygous GC carriers.

| | Genotype | | |
|---|---|---|---|
| | GC/GC | GC/TT | TT/TT |
| Ejection | 27.2 ± 4.9 | 22.4 ± 2.4 | 22.6 ± 3.2 |
| fraction (%) | | | |

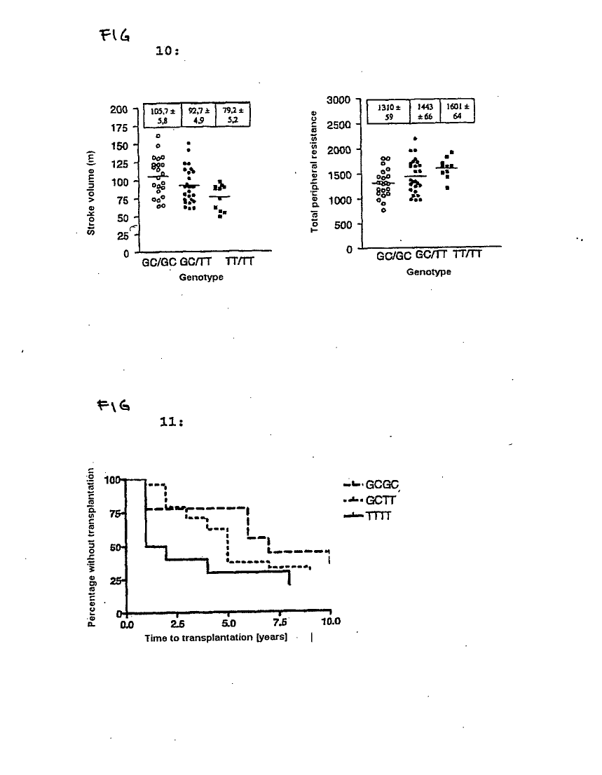

These differences in the ejection performance of the heart are associated with differences in the disease course: The median time to heart transplantation in patients with heart insufficiency due to cardiomyopathy is 7 years in homozygous GC carriers, 5 years in heterozygotes, and only 1.5 years in homozygous TT carriers (FIG. 11).

The Role of GNAQ on the Effects of Insulin and Endothelin in Adipose Tissue

Glucose uptake in adipose tissue is usually regulated through tyrosine phosphorylation of IRS-1, followed by binding to the p85 subunit of PI-3 kinase. However, some studies have also shown a IRS-1-independent glucose transport into the cell which is mediated by insulin or endothelin. Endothelin-mediated glucose uptake depends on Gq, but not on PI-3K (Kanzaki et al., 2000; Wu-Wong et al., 1999; Rachdaoui and Nagy, 2003). TNF alpha is considered to be a critical mediator in insulin resistance (Hotamisligil, 2000), with adipose tissue being one of the main target sites of TNF alpha (Ruan et al., 2002). In adipocytes it was shown that chronic treatment with TNF alpha leads to a reduced endothelin 1-dependent glucose uptake. In addition it led to a decreased Gq/11 expression, and this decreased expression resulted in a decreased glucose uptake into the cell (Rachdaoui and Nagy, 2003). It can thus be assumed that a genotype-dependent modified Gq expression in adipose tissue may result in different insulin responses and pathogenesis of insulin resistance, which may contribute to diseases such as diabetes mellitus type II or polycystic ovary syndrome (PCOS).

Use of Gene Modifications in the GNAQ Gene to Predict the Effect of Insulin in Adipose Tissue

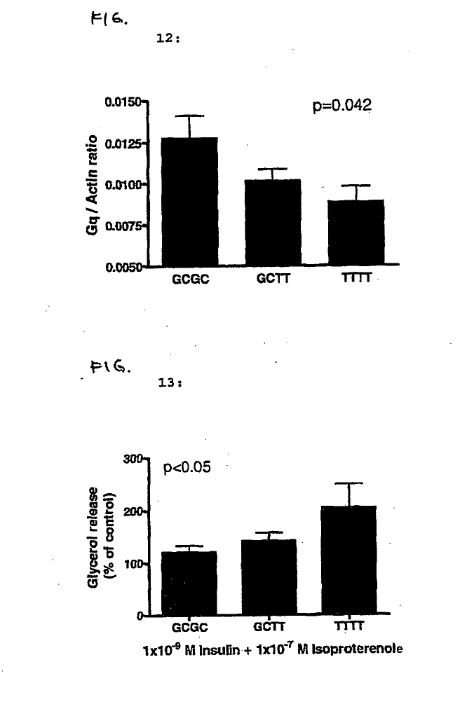

Due to the molecular investigation of the GC(−909/−908)TT polymorphism (stronger activability of the promoter in GC alleles and increased mRNA expression in GCGC genotypes) it could be assumed that the observed effects could be transferred to Gq-mediated effects in adipose tissue. In order to prove this hypothesis, genotype-dependent mRNA expression in human adipose tissue was initially studied. For this, mRNA was isolated from human subcutaneous adipose tissue by mamma reduction plasty, and was transcribed into cDNA by reverse transcriptase. The method is known to the expert. Then, the level of expression was determined by real time-PCR (Taqman procedure), and was compared with the level of expression of the housekeeping gene ?-actin. As shown in FIG. 12, the Gq mRNA expression in human adipose tissue in GC homozygotes is increased by approx. 40% compared to heterozygotes or homozygous TT carriers.

The inhibiting effect of insulin on lipolysis was studied in human adipocyte cultures. For this, lipolysis was induced ex vivo in human adipocytes by isoprenaline, and the inhibiting effect of insulin was studied by adding insulin to the cultures. Lipolysis was quantified by glycerol release (Hauner et al., 2002). Here, the inhibiting insulin effect was strongest with adipocytes from individuals with GCGC genotype (FIG. 13). This coincides with the observation of an increased gene expression of Gq in this genotype. In addition, these results indicate that gene modifications in the GNAQ gene are suitable for prediction of drug efficacy as it has been shown here for insulin as an example.

Insulin Resistance in the Example of PCOS Patients

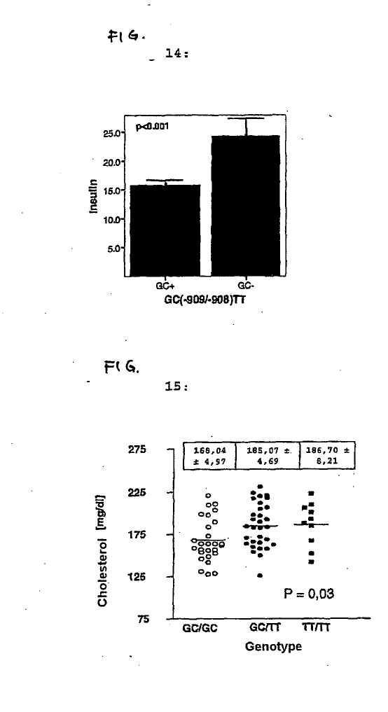

Polycystic ovary syndrome (PCOS) is a frequent endocrine disease which is characterized by chronic anovulation and hyperandrogenism. It affects approx. 5% of all pre-menopausal women. Most women with PCOS have insulin resistance and therefore an increased risk for metabolic syndrome (obesity, type 2 diabetes, lipometabolic disorder and hypertension). The body mass index (BMI) and insulin resistance, measured according to the HOMA IR method (homeostasis model assessment for insulin resistance), were examined with Gq genotypes of the GC(−909/−908)TT polymorphism. FIG. 14 shows clearly that TT/TT-homozygous patients have significantly increased BMI, HOMA-IR and insulin serum concentrations.

It therefore was demonstrated that gene modifications in the GNAQ gene may be used to predict insulin resistance.

Risk of Metabolic Disorders

One of the most frequent metabolic disorders is hypercholes-terinemia which is known to be associated with an increased risk of coronary heart disease, myocardial infarction, stroke and Alzheimer's disease. In young healthy individuals already can be found that the GC(−909/−908)TT polymorphism is associated with a modified cholesterol serum concentration (FIG. 15).

Fundamental Properties of Malignant Tumors

In malignant tumors, also known as cancer, there are characteristic changes in fundamental functions, which support the undesired growth of cells of this sort. Cancer cells are characterized by a loss of contact inhibition and uncontrolled cell growth. Changes of this sort are triggered by many toxins, so-called carcinogens, which damage hereditary material. These toxins include many chemicals, tobacco smoke and UV light. In addition, genetic factors play a dominant role in the origin of cancer. Cancer cells are characterized not only by their uninhibited growth, but also by their tendency to produce daughter growths (metastases) in other organs. Metastases are regularly spread by the circulation of the blood or through lymph vessels. In many cases, cancer is not curable and leads to death. Therapeutic attempts are made to remove the initial tumor and the metastases by operation. In addition, tumors can be irradiated. Using so-called cytostatics, antibodies to certain proteins or cell surface markers or immune modulators (cytokines, interferons), it is attempted to kill the rapidly dividing cancer cells or to move them into programmed cell death (apoptosis). Currently available therapeutic measures only prolong life in most cases, but do not lead to a definite cure.

Prognostic Factors in Cancer

Definition of prognostic factors for the clinical course of cancer is of considerable medical significance. These should provide information on the response to certain forms of therapy or be generally predictive for the occurrence of metastases, tumor progression and survival. Prognostic factors generally known to the medical expert have been used. For example, these include the size of the tumor, its depth of penetration into the surrounding tissue, growth beyond organs, penetration into blood or lymph vessels or lymph nodes or the degree of differentiation of the tumor cells. In addition, there are some relatively unspecific serological markers. The procedure for classifying tumors is generally referred to as “staging” and “grading”. It is generally the case that the present of distant metastases and a low degree of differentiation are very unfavorable prognostic factors. It is nevertheless the general experience in medicine that patients with the same tumor stage can exhibit drastically different clinical courses. In some patients, there is rapid progression of the disease and metastases and relapses occur, in other patients the disease stops developing for unclear reasons. Metastases then can be local, regional or distant from the initial tumor. For this it is necessary that a large number of malignant cells are washed through the lymphatic or blood circulation into neighboring tissue, or passed on by direct contact. A tendency to relapse means the recurrence of a tumor after incomplete or partial operative removal of the tumor. This is not renewed malignant transformation, but regrowth of tumor tissue which had not been fully removed. Relapse from metastases is also possible, as these can remain latent for many years. The term progression means the recurrence of a tumor with higher grading (more de-differentiation) or the reappearance of metastases.

There are very evidently many individual unrecognized biological variables which are major determinants of the clinical course of a tumor disease, unrelated to staging or grading. These factors include genetic host factors.

It is then desirable to develop genetic markers which are predictive for the occurrence of tumors. These markers fulfill the function of assuring that affected individuals are given additional screening measures at an early stage (serology, X-ray, ultrasound, NMR etc.). In this way, cancer can be recognized at an early stage and therapy attempted, thus greatly increasing the chances of cure and survival, as these are much better in early stage than in advanced tumors.

Types of Tumors

In general, all cells in the human body can be malignantly transformed and lead to cancer. The mechanisms of tumor progression, metastasis and therapeutic progression described above and later are generally applicable. These mechanisms and claims therefore apply to all human tumors, in particular to the following tumors:

Tumors of the urogenital tract: in particular, carcinoma of the urinary bladder, prostate carcinoma, renal cell carcinoma and seminoma.

Tumors of the female genital organs: Mammary carcinoma, ovarian carcinoma, cervical carcinoma.

Tumors of the gastrointestinal tract: carcinoma of the oral cavity, esophageal carcinoma, gastric carcinoma, liver carcinoma, liver carcinoma, bile duct carcinoma, pancreatic carcinoma, colon carcinoma, rectal carcinoma.

Tumors of the respiratory tract: larynx carcinoma, bronchial carcinoma.

Tumors of the skin: malignant melanoma, basalioma, T-cell lymphoma

Tumors of the hematopoietic system: Hodgkin's and non-Hodgkin's lymphoma, acute and chronic leukemia etc.

Tumors of the brain or nervous tissue: glioblastoma, neuroblastoma, medulloblastoma, meningeal sarcoma, astrocytoma.

Soft tissue tumors, for example, sarcomas and tumors of the head and neck

G Proteins and Malignant Cell Transformations

in vitro experimental studies show that mutations in G proteins subunits can cause malignant transformations in cells. The expression of a constitutively active Gαq-subunit lacking GTPase-activity leads to malignant transformation of fibroblasts (Kalinec et al., 1992).

G Protein Mutations and Cancer in Man

Somatic mutations in the subunits Gαs and Gαi2 have been detected in some rare adenomas in man. These are designated as Gip2 (Gαi2-subunit)or as Gsp (Gαs-subunit). These are not genetic host factors which modulate the course of the disease, but causal factors (reviewed by: Farfel et al. 1999).

Use of Gene Modifications in the GNAQ Gene to Predict the Course of Tumor Diseases

An important component of the present invention is the provision of diagnostically relevant gene modifications in the GNAQ gene as a prognostic factor for all human tumor diseases. In the nature of the matter, all tumor diseases cannot be described. Therefore, the principle will be described in selected examples, which demonstrate its general applicability:

The following examples are intended to further describe the invention.

### EXAMPLE 1

### Chronic Lymphatic Leukemia (CLL)

Chronic lymphatic leukemia is a chronic form of leukemia. This disease is characterized by a large number of degenerated lymphocytes. A total of 30% of all leukemia cases are chronic lymphatic leukemia. The mean age at which this disease first occurs is 65 years. Only ten percent of the patients are under 50 years old. Men are about two to three times more frequently affected than are women. No risk factors are known for the development of CLL. The disease is nevertheless rare in Japan and China. Even Japanese immigrants into the USA fall extremely rarely ill from CLL. This fact indicates that genetic factors play a role. The therapy depends on the stage of the disease. CLL can show a benign course for up to 20 years. This means that the patient exhibits no symptoms apart from enlarged lymph nodes and possibly tiredness and loss of appetite. Treatment only starts when the number of lymphocytes starts to rise above a certain extent, the proportion of red blood cells and the number of blood platelets sinks or other complications occur. Early treatment has no effect on the course or the result of the disease. Chemotherapy is the most important therapeutic measure. In certain cases, the patient must also be irradiated or operated on. Patients can live up to 20 years with the diagnosis of CLL, without exhibiting severe symptoms. However, the more advanced the disease is, the greater is the damage to health because of modifications to organ systems. The doctor can assess the prognosis of the disease on the basis of the Binet stage. The characteristics of the stage of CLL include the number of lymphocytes in the blood and bone marrow, the size of the spleen and liver and whether the patient is anemic or not. CLL leads to changes in the immune system, so that patients suffering from CLL are more at risk of developing other types of cancer. However, the clinical course of patients with the same Binet stage can be quite disparate. It is a component of this invention to show that gene modifications in the GNAS gene are suited to predict the clinical course of CLL. For this purpose, patients with CLL were genotyped with respect to the described genetic modifications in GNAS and the gene status compared with the progression of the disease. Progression is defined here as the time interval between the first diagnosis of CLL and the necessity for therapy.

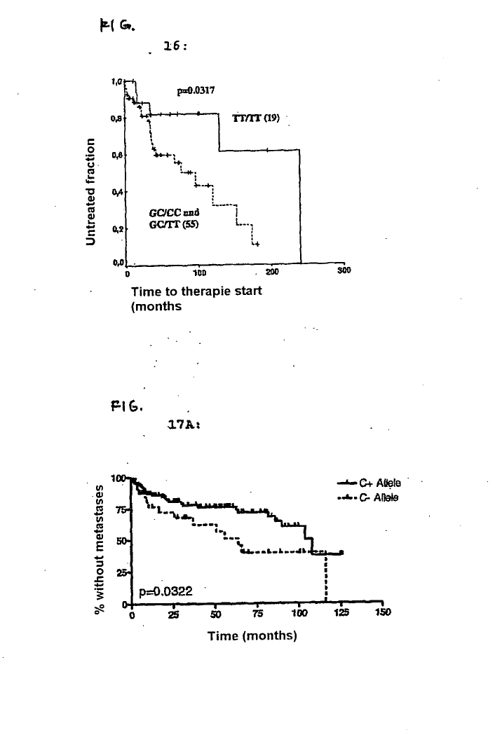

FIG. 16 shows a significant difference between therapeutic freedom depending on the GNAQ GC(−909/−908)TT polymorphism, wherein the CC genotype is associated with a two times faster disease progression (hazard risk 2.07; p=0.02).

### EXAMPLE 2

### Urinary Bladder Carcinoma

Bladder carcinoma is a malignant tumor in the mucous membrane of the bladder. Bladder cancer occurs most often in patients aged between 60 and 70. Men are three times more often affected than women. Bladder cancer is the third most frequent form of cancer in men, after lung cancer and prostate cancer. Bladder cancer can be caused by external influences. The risk factors include smoking, continuous stress to the organism from chemicals, such as dyes or analgesic abuse. Investigation of many patients shows that the tumor is superficial. This can be removed operatively with the help of a cystoscope. More than 70% of the patients treated for a superficial bladder carcinoma develop a recurrent tumor later. More than half of the recurrent tumors are non-muscle invasive. These can be treated or controlled by transurethral resection. It is therefore important to recognize the lesions early and to monitor the patient regularly and closely. The most important control is cystoscopy with urine cytology. Regular elimination urograms serve to control possible manifestations of the tumor in the renal pelvis and ureters. There has hardly been any valid marker which is predictive for the subsequent course of the disease. Classical factors such as depth of penetration, degree of differentiation, metastasis, lymph node involvement, etc., are therefore currently used for prognosis. Genetic markers for tumor progression, tendency for relapses, probability of survival and response to therapy would bring a major improvement in the care of patients with bladder carcinoma.

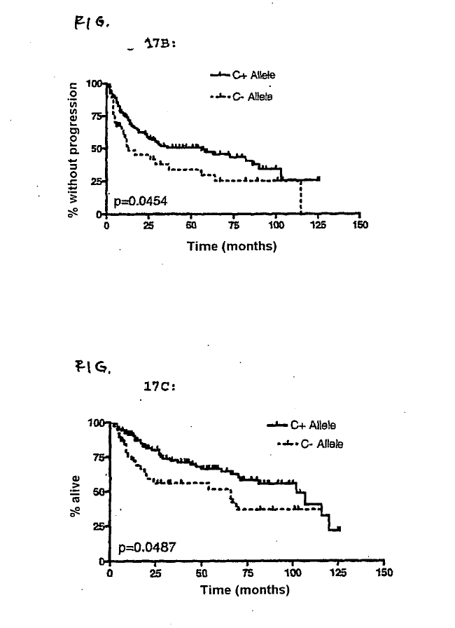

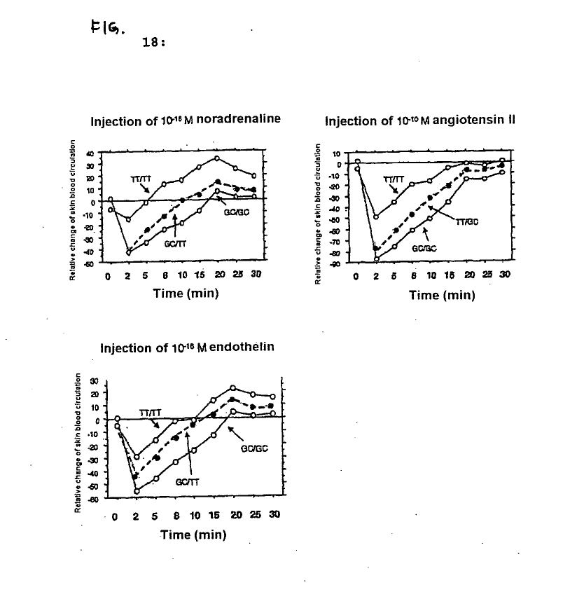

According to the invention gene modification in GNAQ are used for the prediction of the further clinical course of the disease. FIG. 17A shows the time point up to the occurrence of metastases, depending on the GC(−909/−908)TT polymorphism. The risk of metastasis in patients with the C allele is increased by a factor of approximately 2. The median time till metastasis is 108 months with the GC/TT and the GC/CG genotypes, but 64 months with the TT/TT genotype. A similar relationship is found if the time up to tumor progression is examined (FIG. 17B). Progression is understood as the occurrence of metastases or the recurrence of the tumor with higher staging or grading. The shape of the curve is significantly different for the genotypes (p=0.045, Log rank test), where the GC genotypes are assigned to the less favorable clinical course. Finally, the connection between the GNAQ GC(−909/−908)TT polymorphism and survival is depicted (FIG. 17C). Here too it is evident that patients with the GNAQ genotypes TT//TT die earlier than patients with the GC allele. The median time of survival is 102 months with the GC genotype, but only 66 months for the combined TT/TT genotype.

Use of Gene Modifications in the GNAQ Gene to Predict Risks of Disease and Clinical Courses

As the wide variety of functions of Gαq is well known, gene modifications in the GNAQ gene can increase the risk for many different diseases or influence the clinical courses. It is not possible in principle to investigate all human diseases and their clinical courses. However, we have taken three different diseases as examples: left heart hypertrophy, CLL and bladder carcinoma. These data prove unambiguously that gene modifications in the GNAQ gene can be used for the purpose described here. There is no a priori connection between these diseases.

Pharmacogenetics—Diagnosis of the Efficacy of Drugs, their Potency and the Occurrence of Adverse Effects

Principles and Goals of Pharmacogenetics

The efficacy of drugs and/or the occurrence of side-effects are defined by a series of parameters, aside from the specific chemical properties of the chemically defined products. Two .important parameters, the maximal plasma concentration and the plasma half-life, determine the efficacy or lack of efficacy or the occurrence of side-effects to a large extent. Factors determining the plasma half-life include the rate of metabolism of drugs in the liver or other body organs to active or inactive metabolites and the speed of elimination from the body, which can be through the kidneys, through respiratory air, through sweat, through seminal fluid, through stools or through other body secretions. The efficacy after oral administration is limited by the so-called “first-pass effect”, as, after absorption of a drug from the intestine, a defined proportion is metabolized in the liver to inactive metabolites.

Mutations or polymorphisms in the genes of metabolizing enzymes can modify their activity by modifying their aminoacid composition in such a way that the affinity to the substrate to be metabolized can be increased or decreased, so that metabolism is accelerated or slowed down. In a similar manner, mutations or polymorphisms in transport proteins can modify their amino acid composition in such a way that the transport and thus elimination from the body is accelerated or slowed down.

In the selection of the optimally suited substance for a patient, the optimal dosage, the optimal formulation and for the avoidance of undesired side-effects—which can damage health or be fatal—the knowledge of genetic polymorphisms or of mutations which lead to modification of the gene products is extraordinarily important.

The Action of Hormones in the Human Body and the Significance of Polymorphisms in Hormone Receptors

Many hormones and peptide hormones of the human body and receptor antagonists exert their activity by acting on the so-called receptors in body cells. These are proteins of various structures. After activation of these receptors, the signals must be transmitted into the interior of the cell, which is mediated by activation of heterotrimeric G proteins. Proteins of this type are composed of different α-, β- and γ-subunits. These receptors can be classified into certain groups, depending on their activability by defined hormones. The expert is aware that mutations or polymorphisms in certain receptors can determine the efficacy of certain agonists or antagonists on these receptors. Thus a frequent Gly16Arg-polymorphism in the gene that codes for the β₂-adrenoceptor influences the strength of response to the β₂-sympathomimetic drug salbutamol (Martinez et al. 1997). Polymorphisms in the D₂-receptor gene determine the frequency of the occurrence of dyskinesia in the treatment of Parkinson's disease with levadopa (Oliveri et al. 1999). Polymorphisms in the μ-opiate receptor gene determine the analgesic efficacy of opiates (Uhl et al. 1999).

These gene modifications in specific receptors can only be used in the diagnosis of the actions of drugs to the extent that these drugs are specific agonists or antagonists of the receptors under consideration. On the other hand, it would be desirable to have individual diagnosis of the general response to all drugs and the individual prediction of the risk of adverse effects under therapy with drugs.

Diagnosis of the Activability of G Proteins Permits General Diagnosis of the Efficacy of Drugs, their Optimal Dosage and the Occurrence of Side-Effects

The expression “drug” generally means substances which are added to the human body from the outside, in order to produce defined states. These substances can be hormones, low or high molecular weight substances, peptides or proteins, antibodies or many others. Most of the drugs used to treat diseases, physical malfunction or impairment in well-being, are hormones, agonists on hormone receptors or other substances which directly or indirectly influence the expression of receptors or the concentration of hormones. A series of drugs exert their influence in that, during therapy with such substances, physiological counter-regulation takes place which raises the concentration of hormones which activate the G protein-coupled receptors. A generally known example which may be mentioned is treatment with diuretics, in particular, loop diuretics and thiazide diuretics. The loss of sodium chloride occurring during therapy leads to the activation of the renin-angiotensin-aldosterone systems. The increased levels of the hormone angiotensin II formed stimulate increased absorption of sodium in the kidney, stimulate salt uptake, increase blood pressure through a direct vasoconstrictory effect on the cells of vascular smooth muscle and induce proliferation processes. It is generally known that these mechanisms evoked by angiotensin II occur after coupling of the hormone to receptors which mediate their activity through activation of heterotrimeric G proteins. The efficiency of these actions is predictable if the strength of the activability of G proteins can be diagnosed. Other drugs exert their activity by inhibiting the re-uptake of transmitters released from neurones, e.g. noradrenaline, adrenaline, serotonin or dopamine. As an example, the drug sibutramine may be given, which inhibits the re-uptake of serotonin and noradrenaline in the central nervous system, as a consequence of which the feeling of hunger is reduced and thermogenesis is increased. Corresponding to this, sibutramine can be used for the therapy of obesity. As noradrenaline and serotonin activate G protein-coupled receptors, the diagnosis of the activability of G proteins is particularly well suited for the prediction of the efficacy of sibutramine and the occurrence of typical, sibutramine-associated side-effects (e.g. increase in heart rate and blood pressure).

The invention is based on the fact that a procedure was invented which is generally suitable for the diagnosis of the activability of G proteins. For this purpose, one or several polymorphisms in the GNAQ gene are investigated that code for the human Gαq-subunit of heterotrimeric G proteins. Those polymorphisms are particularly suited which predict the diagnosis or the non-occurrence of an alternative splice procedure of the gene or modified expression of Gαq. With overexpression, there is predictably increased activability of hetrotrimeric G proteins and increased activability of all cells of the human body. Thus determination of the presence of polymorphisms in GNAQ permits the diagnosis of the efficacy and adverse effects of drugs, in particular, agonists and antagonists of all receptors of which the activity is mediated through heterotrimeric G proteins. In addition, those polymorphisms in GNAQ can be used to diagnose the actions of drugs which, either indirectly or as a consequence of counterregulatory mechanisms of the body, raise or lower the concentrations of endogenous hormones, of which the receptors activate hetrotrimeric proteins. Thus the invention allows the diagnosis of actions and side-effects of all drugs and is not limited to drugs which influence specific receptors in an agonistic or antagonistic manner. In addition, the diagnosis of the allelic or haplotype status in GNAQ can be used to determine the individual optimal and tolerated dosage of drugs.

For the diagnosis or increased or reduced activability of G proteins, detection of the GC(−909/−908)TT polymorphism is particularly suitable, either alone or in all possible combinations. In addition, all other gene modifications in GNAQ can be used for diagnosis which are in coupling disequilibrium to these polymorphisms and/or also increase or decrease the alternative splice process or the expression.

These gene modifications can be detected with any procedure known to the expert, e.g. direct sequencing, restriction analysis, reverse hybridization, dot-blot or slot-blot procedure, mass spectrometry, Taqman or light cycler technology, pyrosequencing etc. In addition, these gene polymorphisms can be simultaneously detected on a DNA chip after multiplex-PCR and hybridization. In addition, for the diagnosis of increased activability of G proteins, other procedures can be used which permit the direct detection of the level of expression of Gαq or of the splice variants of Gαq.

The described procedure is particularly suitable for the diagnosis of the activity of agonists or antagonists on receptors the activity of which is known to be mediated by G proteins. The following examples are named for this purpose and this list of examples could be extended:
1. Adrenergic receptors, in particular α- and β-adrenoceptors and their isoforms and subgroups, i.e. α₁- and α₂-adrenoceptors and β₁-, β₂-, β₃- and β₄-adrenoceptors
2. Muscarinic receptors and their isoforms, e.g. m₁-, m₂-, m₃-, m₄- and m₅-muscarinic receptors and their subtypes. Typical antagonists on muscarinic receptors are, for example, atropine, scopolamine, ipratroprium, pirenzepine and N-butylscopolamine. Typical agonists are carbachol, bethanechol, pilocarpine etc.
3. Dopamine receptors, e.g. D₁-, D₂-, D₃-, D₄-, and D₅-receptors and their isoforms and splice variants
4. Serotonin receptors, e.g. 5-HT₁- 5-HT₂-, 5-HT₃-, 5-HT₄-, 5HT₅-, 5HT₆- und 5-HT₇-receptors and their subtypes, isoforms and splice variants. Typical agonists are sumatriptan and cisapride; antagonists are for example ondansetrone, methysergide, buspirone and urapidil.
5. Endothelin receptors and their subtypes, isoforms and splice variants
6. Bradykinin receptors, e.g. B₁- and B₂-receptors and their subtypes, isoforms and splice variants
7. Angiotensin receptors, e.g. AT II type 1 and type 2 receptors; typical antagonists on the AT II-receptor are losartan and other sartans.
8. Receptors for endorphins and opiates, e.g. the μ-opiate receptor
9. Chemokine receptors CCR1-12 and CXCR1-8 for e.g. interleukin-1/2/3/4/5/6/7/8/9/10/11/12, RANTES, MIP-1α, MIP-1β, stromal cell-derived factor, MCP1-5, TARC, lymphotactin, fractalkine, eotaxin 1-2, NAP-2, LIX etc.
10. Adenosine receptors and their subtypes, isoforms and splice variants
11. Receptors for thrombin (protease-activated receptors)
12. Receptors for lyso-phophatidic acid, phosphatidic acid; receptors for sphingosine phosphate and their derivatives
13. Receptors for prostaglandins and thromboxanes, e.g. for PGE1, PGE2, PGF, PGD2, PGI2, PGF2α, thromboxane A2, etc.
14. Receptors for neuropeptides, e.g. NPY1-5
15. Histamine receptors, e.g. H₁- and H₃-receptors
16. Receptors for platelet-activating factor (PAF-receptor)
17. Receptors for leukotrienes
18. Receptors for insulin, glucagon, insulin-like growth factor (IGF-1 and IGF-2), epidermal growth factor (EGF) and platelet-derived growth factor (PDGF)
19. Receptors for growth hormone (GH), somatostatin (SSTR1-5), thyreotropic hormone (TSH), oxytocin, prolactin, gonadotropins
20. Receptors for cytokines, e.g. interferons
21. Receptors for purines
22. Orphan receptors, of which the activity is mediated by G proteins
23. Receptors for leptin
24. CpG—Oligonucleotide

Moreover, the activity can be predicted of drugs which influence the re-uptake, metabolism or de novo synthesis of neurotransmitters or, during therapy with which, there are changes in the expression or sensitivity of the above named receptors (e.g. sibutramine, fluoxetine). In addition, the actions of all drugs can be diagnosed which directly, indirectly or as the consequence of a physiological counter-regulation, change the concentrations of agonists which activate the above receptors. The effect of radiation therapy on cancer patients can also be predicted.

In particular, the actions and side-effects of the following drugs from the following areas of indication can be diagnosed:
1. Antihypertensive drugs, e.g. β-blockers (propanolol, bis-prolol, etc.), diuretics (hydrochlorothiazide and other thiazide diuretics; furosemide, piretanide and other loop diuretics, chlorthalidone), α₁-adrenoceptorblockers (e.g. doxazosin, prazosin), angiotensin receptor blockers (e.g. losartan), ACE-inhibitors(enalapril, captopril, ramipril etc.), Ca²⁺-channel blockers (e.g. nifedipine, verapamil, amlodipine, felodipine), clonidine, reserpine, renin- inhibitors
2. Drugs for the treatment of heart failure, e.g. β-blockers (e.g. propanolol, metoprolol), ACE-inhibitors (e.g. captopril, enalapril, ramipril, etc.), angiotensin receptor blockers (e.g. losartan), digitalis glycosides, catecholamines, diuretics.
3. Drugs for the treatment of low blood pressure or heart failure, e.g. α- and β-sympathomimetics (effortil, adrenaline, noradrenaline, dobutamine, β-adrenoceptor blockers, ACE-inhibitors, angiotensin II-receptor blockers.)
4. Drugs for the treatment of migraine, e.g. sumatriptan, rizatriptan, zolmitriptan and other agonists for serotonin receptors, β-blockers (propanolol, timolol), ergotamine and dihydroergotamine
5. Analgesics of the morphine type (morphine, codeine, etc.)
6. Drugs for the treatment of coronary heart disease, such as adenosine, β-blockers (e.g. propanolol, acebutolol), nitrates and Ca²⁺-channel blockers
7. Drugs for the treatment of psychiatric diseases (schizophrenia, manic-depressive diseases, psychoses, depression) and addictive disease, such as alcoholism, (e.g. fluoxetine, paroxetine, imipramine, desipramine, doxepin, mianserin, trazodone, lofepramine), anxiety syndrome (diazepam, etc.), which, for example, influence the dopaminergic, serotoninergic or adrenergic systems. Also drugs which act through receptors for GABA, glycine or glutamate or their derivatives.
8. Drugs for the treatment of Alzheimer's disease (e.g. tacrine) and for the treatment of Parkinson's disease (e.g. bromocriptine, L-DOPA, carbidopa, biperidene, seleginil, etc.) which influence transmitter concentrations of, e.g. muscarinergic or dopaminergic substances.
9. Drugs for the treatment of bronchial asthma, which, for example, either possess direct bronchodilatory activity or antiinflammatory activity, e.g. salbutamol, terbutaline, albuterol, theophylline, montelukast, zafirlukast, cromoglycic acid, ipratropium bromide. This group of drugs also includes antibodies to certain proteins and receptors.
10. Drugs for the treatment of disturbances in the motility of the stomach or intestine and drugs for the treatment of irritable bowel syndrome, e.g. N-butylscopolamine, pirenzepine, metoclopramide)
11. Drugs for the treatment of obesity, which either directly activate receptors with lipolytic activity, e.g. β₃-adrenergic agonist, or are centrally active, e.g. sibutramine, or similar substances which alter satiety or which influence thermogenesis. This also includes drugs which influence gastric emptying.
12. Drugs for the treatment of chronic inflammatory processes or disturbances in the immune system, e.g. cytokines (interferons) in the therapy of virus hepatitis or interleukin-2 in HIV infection. These diseases also include Crohn's disease, ulcerative colitis, asthma, psoriasis, neurodermitis, hay fever. This also includes antibodies to cytokines or to cytokine receptors, e.g. to TNFα
13. Drugs for the treatment of gestosis, pre-eclampsia/eclampsia and the HELPP syndrome.
14. Drugs for the treatment of disturbances in fertility or to rectify menstrual abnormalities in the women or for contraception.
15. Drugs for the treatment of cardiac arrythmias
16. Antidiabetic drugs (acarbose, insulin, troglitazone, metformin, etc.)
17. Hypnotics, antiemetics and antiepileptic drugs
18. Drugs for the treatment of disturbances in sexual function, e.g. erectile dysfunction, female sexual dysfunction, lack of libido, disturbances in orgasm (phosphodiesterase inhibitors such as sildenafil, prostaglandin E1, agonists to dopamine receptors, e.g. apomorphine, yohimbine, phentolamine)
19. Drugs for the treatment of cancer and chemotherapeutic drugs, e.g. 5-fluoruracil, antibodies to proteins and receptors (e.g. to HER-2), substances which block tyrosine kinases etc.
20. Drugs for the treatment of allergic and tumor diseases, in which the effect is achieved by administration of CpG-nucleotides.
21. Drugs for the treatment of obesity, the metabolic syndrome or diabetes, e.g. sibutramine, orlistat, leptin, topiramate, glinide, glitazone, biguanide etc.
22. Drugs for the treatment of the HIV-infection, including antibodies and receptor blockers. Prediction of the occurrence of lipodystrophy during treatment with proteinase inhibitors.

It is of course not possible in the context of the invention described here to prove that all drug actions are determined by the GNAQ gene status. In the nature of things, it is also impossible to investigate the genotype-dependent actions of drugs which will only be developed and used in future. On the contrary, examples of drugs are presented with a range of different mechanisms of actions, so that these findings can be generalized.

As an example for the general usability of the GNAQ GC(−909/−908)TT polymorphism to predict drug actions, the vasoconstrictors angiotensin II, noradrenaline or endothelin were injected under the skin of human individuals, and the following change of blood circulation was recorded (FIG. 18). It can be seen after administration of all three hormones coupling to different receptors that the reduction of skin blood circulation is highest for the GC/GC genotype, which may be explained by the increased expression of G?q.

Another proof for the general suitability of gene modifications in the GNAQ gene to predict drug actions results from the observed genotype-dependent disease courses in patients with bladder carcinoma. All of these patients were treated with different drugs. The individual disease courses which were made visible by the use of gene modifications in the GNAQ gene, support a differential response to these therapy forms.

### REFERENCES

Adams, J. W. and Brown, J. H. (2001). G-proteins in growth and apoptosis: lessons from the heart. Oncogene 20, 1626-1634.

Bourne, H. R. (1997). How receptors talk to trimeric G proteins. Curr. Opin. Cell Biol. 9, 134-142.

De, V. M., Chen, J., Codina, J., and Iyengar, R. (1992). Enhanced phospholipase C stimulation and transformation in NIH-3T3 cells expressing Q209LGq-alpha-subunits. J. Biol. Chem. 267, 18263-18266.

Farfel, Z., Bourne, H. R., and Iiri, T. (1999). The expanding spectrum of G protein diseases. N. Engl. J. Med. 340, 1012-1020.

Fernandez-Real, J. M., Penarroja, G., Richart, C., Castro, A., Vendrell, J., Broch, M., Lopez-Bermejo, A., and Ricart, W. (2003). G protein beta3 gene variant, vascular function, and insulin sensitivity in type 2 diabetes. Hypertension 41, 124-129.

Givertz, M. M., Colucci, W. S., and Braunwald, E. (2004). Clinical aspects of heart failure. In Braunwald's Heart Disease: a textbook of cardiovascular medicine, D. P. Zipes, P. Libby, R. O. Bonow, and E. Braunwald, eds. (Philadelphia: Elsevier Saunders), pp. 539-568.

Hauner, H., Meier, M., Jockel, K. H., Frey, U. H., and Siffert, W. (2003). Prediction of successful weight reduction under sibutramine therapy through genotyping of the G-protein beta3 subunit gene (GNB3) C825T polymorphism. Pharmacogenetics 13, 453-459.

Hauner, H., Rohrig, X., and Siffert, W. (2002). Effects of the G-protein beta3 subunit 825T allele on adipogenesis and lipolysis in cultured human preadipocytes and adipocytes. Horm. Metab Res. 34, 475-480.

Hegele, R. A., Anderson, C., Young, T. K., and Connelly, P. W. (1999). G-protein beta3 subunit gene splice variant and body fat distribution in Nunavut Inuit. Genome Res. 9, 972-977.

Hotamisligil, G. S. (2000). Molecular mechanisms of insulin resistance and the role of the adipocyte. Int. J. Obes. Relat Metab Disord. 24 Suppl 4, S23-S27.

Iiri, T. and Bourne, H. R. (1998). G proteins propel surprise. Nat. Genet. 18, 8-10.

Iiri, T., Farfel, Z., and Sourne, H. R. (1998). G-protein diseases furnish a model for the turn-on switch. Nature 394, 35-38.

Kalinec, G., Nazarali, A. J., Hermouet, S., Xu, N., and Gutkind, J. S. (1992). Mutated alpha subunit of the Gq protein induces malignant transformation in NIH 3T3 cells. Mol. Cell Biol. 22, 4687-4693.

Karnzaki, M., Wateon, R. T., Artemyev, N. O., and Pessin, J. B. (2000). The trimeric GTP-binding protein (G(q)/G(11)) alpha subunit is required for insulin-stimulated GLUT4 translocation in 3T3L1 adipocytes. J. Biol. Chem. 275, 7167-7175.

Lindemann, M., Barsegian, V., Siffert, W., Perencik, S., Roggendorf, M., and Grosse-Wilde, H. (2002). Role of G protein beta3 subunit C825T and HLA class II polymorphisms in the immune response after HBV vaccination. Virology 297, 245-252.

Martinez, F. D., Graves, P. E., Baldini, M., Solomoon, S., and Erickson, R. (1997). Association between genetic polymorphisms of the beta2-adrenoceptor and response to albuterol in children with and without a history of wheezing. J. Clin. Invest 100, 3184-3188.

Mende, U., Kagen, A., Conen, A., Aramburu, J., Schoen, F. J., and Neer, E. J. (1998). Transient cardiac expression of constitutively active Galphaq leads to hypertrophy and dilated cardiomyopathy by calcineurin-dependent and independent pathways. Proc. Natl. Acad. Sci. U.S.A 95, 13893-13898.

Mitchell, A., Buhrman, S., Seifert, A., Nurnberger, J., Wenzel, R. R., Siffert, W., Philipp, T., and Schafers, R. F. (2003). Venous response to nitroglycerin is enhanced in young, healthy carriers of the 825T allele of the G protein beta3 subunit gene (GNB3). Clin. Pharmacol. Ther. 74, 499-504.

Molkentin, J. D. and Dorn II, G. W. (2001). Cytoplasmic signaling pathways that regulate cardiac hypertrophy. Annu. Rev. Physiol 63, 391-426.

Morrison, A. C., Doris, P. A., Folsom, A. R., Nieto, F. J., and Boerwinkle, E. (2001). G-protein beta3 subunit and alpha-adducin polymorohisms and risk of subclinical and clinical stroke. Stroke 32, 822-829.

Naber, C. K., Husing, J., Wolfhard, U., Erbel, R., and Siffert., W. (2000). Interaction of the ACE D allele and the GNB3 825T allele in myocardial infarction. hypertension 36, 986-989.

Nuckel, H., Frey, U., Aralh, N., Durig, J., Duhrsen, U., and Siffert, W. (2003). The CC genotype of the C825T polymorphism of the G protein beta3 gene (GNB3) is associated with a high relapse rate in patients with chronic lymphocytic leukaemia. Leuk. Lymphoma 44, 1739-1743.

Nurnberger, J., Dammer, S., Mitchell, A., Siffert, W., Wenzel, R. R., Gossl, M., Philipp, T., Michel, M. C., and Schafers, R. F. (2003a). Effect of the C825T polymorphism of the G protein beta 3 subunit on the systolic blood pressure-lowering effect of clonidine in young, healthy male subjects. Clin. Pharmacol. Ther. 74, 53-60.

Nurnberger, J., Dammer, S., Philipp, T., Wenzel, R. R., and Schafers, R. F. (2003b). Metabolic and haemodynamic effects of oral glucose loading in young healthy men carrying the 825T-allele of the G protein beta3 subunit. Cardiovasc. Diabetol. 2, 7.

Oliveri, R. L., Annesi, G., Zappia, M., Civitelli, D., Montesanti, R., Branca, D., Nicoletti, G., Spadafora, P., Pasqua, A. A., Cittadella, R., Andreoli, V., Gambardella, A., Aguglia, U., and Quattrone, A. (1999). Dopamine D2 receptor gene polymorphism and the risk of levodopa-induced dyskinesias in PD. Neurology 53, 1425-1430.

Rachdaoui, N. and Nagy, L. S. (2003). Endothelin-1-stimulated glucose uptake is desensitized by tumor necrosis factor-alpha in 3T3-L1 adipocytes. Am. J. Physiol Endocrinol. Metab 285, E545-E551.

Rosskopf, D., Frey, U., Eckhardt, S., Schmidt, S., Ritz, E., Hofmann, S., Jaksch, M., Muller, N., Husing, J., Siffert, W., and Jocke, K. H. (2000). Interaction of the G protein beta 3 subunit T825 allele and the IRS-1 krg972 variant in type 2 diabetes. Eur. J. Med. Res. 5, 484-490.

Rosskopf, D., Manthey, I., and Siffert, W. (2002). Identification and ethnic distribution of major haplotypes in the gene GNB3 encoding the G-protein beta3 subunit. Pharmacogenetics 12, 209-220.

Ruan, H., Miles, P. D., Ladd, C. M., Ross, K., Golub, T. R., Olefsky, J. M., and Lodish, H. F. (2002). Profiling gene transcription in vivo reveals adipose tissue as an immediate target of tumor necrosis factor-alpha: implications for insulin resistance. Diabetes 51, 3176-3188.

Ryden, M., Faulds, G., Hoffstedt, J., Wennlund, A., and Arner, P. (2002). Effect of the (C825T) Gbeta(3) polymorphism on adrenoceptor-mediated lipolysis in human fat cells. Diabetes 51, 1601-1608.

Schafers, R. F., Nurnberger, J., Rutz, A., Siffert, W., Wenzel, R. R., Mitchell, A., Philipp, T., and Michel, M. C. (2001). Haemodynamic characterization of young normotensive men carrying the 825T-allele of the G-protein beta3 subunit. Pharmacogenetics 11, 461-470.

Serretti, A., Lorenzi, C., Cusin, C., Zanardi, R., Lattuada, E., Rossini, D., Lilli, R., Pirovano, A., Catalano, M., and Smeraldi, E. (2003). SSRIs antidepressant activity is influenced by G beta 3 variants. Eur. Neuropsychopharmacol. 13, 117-122.

Siffert, W., Naber, C., Walla, M., and Ritz, E. (1999). G protein beta3 subunit 825T allele and its potential association with obesity in hypertensive individuals. J. Hypertens. 17, 1095-1098.

Siffert, W., Rosskopf, D., Siffert, G., Busch, S., Moritz, A., Erbel, R., Sharma, A. M., Ritz, E., Wichmann, H. E., Jakobs, K. H., and Horsthemke, B. (1998). Association of a human G-protein beta3 subunit variant with hypertension. Nat. Genet. 18, 45-48.

Sperling, H., Eisenhardt, A., Virchow, S., Hauck, E., Lenk, S., Porst, H., Stief, C., Wetterauer, U., Rubben, R., Muller, N., and Siffert, W. (2003). Sildenafil response is influenced by the G protein beta 3 subunit GNB3 C825T polymorphism: a pilot study. J. Urol. 169, 104B-1051.

Spiegel, A. M. (1996). Defects in G protein-coupled signal transduction in human disease. Annu. Rev. Physiol 58, 143-170.

Spiegel, A. M. (1997). Inborn errors of signal transduction: mutations in G proteins and G protein-coupled receptors as a cause of disease. J. Inherit. Metab Dis. 20, 113-121.

Spiegel, A. M. (1999). Hormone resistance caused by mutations in G proteins ana G protein-coupled receptors. J. Pediatr. Endocrinol. Metab 12 Suppl 1, 303-309.

Turner, S. T., Schwartz, G. L., Chapman, A. B., and Boerwinkle, E. (2001). C825T polymorphism of the G protein beta(3)-subunit and antihypertensive response to a thiazide diuretic. Hypertension 37, 739-743.

Uhi, G. R., Sora, I., and Wang, Z. (1999). The mu opiate receptor as a candidate gene for pain: polymorphisms, variations in expression, nociception, and opiate responses. Proc. Natl. Acad. Sci. U. S. A 96, 7752-7755.

Wenzel, R. R., Siffert, W., Bruck, H., Philipp, T., and Schafers, R. F. (2002). Enhanced vasoconstriction to endothelin-1, angiotensin II and noradrenaline in carriers of the GNB3 825T allele in the skin microcirculation. Pharmacogenetics 12, 489-495.

Thu-Wong, J. R., Berg, C. E., Wang, J., Chiou, W. J., and Fissel, B. (1999). Endothelin stimulates glucose uptake and GLUT4 translocation via activation or endothelin ETA receptor in 3T3-L1 adipocytes. J. Biol. Chem. 274, 8103-8110.

Zill, P., Baghai, T. C., Zwanzger, P., Schule, C., Minov, C., Riedel, M., Neumeier, K., Rupprecht, R., and Bondy, B. (2000). Evidence for an association between a G-protein beta3-gene variant with depression and response to anti-depressant treatment. Neuroreport 11, 1893-1897.

1-11. (canceled)

12. A process to identify the disease risk, the course of disease, drug actions, drug side-effects and drug targets which are associated with a base substitution in the GNAQ gene encoding the Gαq-subunit of human G proteins, comprising identifying a base exchange (polymorphism) in the 5′ non-translated region of the gene for the Gαq-subunit of human G proteins.

13. The process of claim 12, wherein a GC(−909/−908)TT polymorphism is identified.

14. The process of claim 12, wherein a G(−382)A polymorphism is identified.

15. The process of claim 12, wherein a G(−387)A polymorphism is identified.

16. The process of claim 12, wherein the presence of two or three of the polymorphisms GC(−909/−908)TT, G(−382)A or G(−387)A are identified.

17. The process of claim 12, wherein the GNAQ gene is sequenced in order to identify one or more of these GNAQ gene polymorphisms.

18. The process of claim 12, wherein the polymorphism in the GNAQ gene is examined or detected by reverse hybridization.

19. The process of claim 12, wherein the polymorphism in the GNAQ gene is examined or detected by restriction enzyme cleavage.

20. The process of claim 17, wherein prior to sequencing, a gene fragment of the GNAQ gene containing one or more of the positions −909, −908, −382, and −387 is amplified.

21. A gene test, comprising a probe for identification of one or more polymorphisms in the 5′ non-translated region of the GNAQ gene.

22. The gene test of claim 21, wherein a probe for identification of one or more of the polymorphisms GC(−909/−908)TT, G(−382)A or G(−387)A is present.
